(19) United States
(12) Patent Application Publication
(10) Pub. No.: US 2026/0132418 A1
(43) Pub. Date: May 14, 2026

(54) GENIC MALE STERILITY IN WATERMELON

(71) Applicant: RIJK ZWAAN ZAADTEELT EN ZAADHANDEL B.V., De Lier (NL)

(72) Inventor: Nelly VICENTE DÓLERA, De Lier (NL)

(21) Appl. No.: 19/333,486

(22) Filed: Sep. 19, 2025

Related U.S. Application Data
(63) Continuation-in-part of application No. PCT/EP2024/057464, filed on Mar. 20, 2024.

(30) Foreign Application Priority Data
Mar. 21, 2023 (WO) PCT/EP2023/057252

(51) Int. Cl. C12N 15/82

(52) U.S. Cl. CPC C12N 15/8289; C12N 15/821

(57) ABSTRACT

A watermelon plant having a modified Male Meiocyte Death 1-like (MMD1-like) gene, the wild type of which comprises the nucleotide sequence of SEQ ID NO. 2, encoding a MMD 1-like protein comprising one or more modifications in the wild type amino acid sequence of SEQ ID NO. 1, or in an amino acid sequence having at least 90% sequence identity to SEQ ID NO. 1 is provided. When the modified MMD1-like gene is homozygously present, the resulting plant exhibits genic male sterility. Also provided are methods for producing and identifying watermelon plants having the modified MMD 1-like gene. Finally, also provided is a method for imparting genic male sterility a watermelon plant comprising introducing a modification to a MMD1-like gene.

### RELATED APPLICATIONS AND INCORPORATION BY REFERENCE

This application is a continuation-in-part application of international patent application Serial No. PCT/EP2024/057464 filed Mar. 20, 2024, which published as PCT Publication No. WO 2024/194361 A1 on Sep. 26, 2024, which claims benefit of international patent application Serial No. PCT/EP2023/057252 filed Mar. 21, 2023.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE STATEMENT

The instant application contains a Sequence Listing which has been submitted electronically and is hereby incorporated by reference in its entirety. Said XML copy was created Sep. 15, 2025, is named Y7954_00664SL.xml and is 10,238 bytes in size.

### FIELD OF THE INVENTION

The present invention relates to a modified gene that confers genic male sterility to a plant, in particular a watermelon plant. The invention further relates to plants comprising the modified gene, to markers capable of detecting the modified gene and to methods and uses involving the modified gene and the markers.

### BACKGROUND OF THE INVENTION

In commercial plant breeding the production of hybrid seed is very important. Plants grown from hybrid seed are generally very uniform, and they benefit from heterosis (hybrid vigour), which can lead to a significant increase in yield and/or performance when compared to the parental lines of the hybrid, or to outcrossing (open-pollinated) lines. Typically, the parental lines used for hybrid seed production are inbred, which implies that their genomes are largely homozygous. The combination of two largely homozygous genomes into a hybrid leads to a high degree of heterozygosity, if both parental lines were genetically unrelated or not closely related.

Efficient hybrid seed production in plant species that are able to self-fertilise requires adequate measures to prevent self-fertilisation of the plants on which hybrid seeds are to be produced. Various strategies have been developed to achieve this, and to obtain an efficient hybrid seed production setup. However, the complexity and amount of labour required for each of these strategies varies greatly.

A strategy that naturally occurs in certain plant species is the physical separation of male and female reproductive organs in separate flowers, either on separate plants (dioecious species) or on the same plant (monoecious species). This system naturally promotes outcrossing, and it can be easily taken advantage of for hybrid seed production.

Another natural strategy is self-incompatibility, which has e.g. been extensively studied in Brassica species. In this case, pollen is physically unable to fertilise egg cells from the same plant. The precise mechanism of the incompatibility interaction can differ. Either pollen hydration or germination is prevented, or pollen tube growth through the style is inhibited by the female tissues, or the pollen tube is not attracted to ripe ovules, or the sperm nuclei are unable to merge with the egg cell nucleus to form a viable zygote. Again, this naturally occurring system is very efficient and useful for preventing self-fertilisation, and for promoting outcrossing.

Another method for preventing selfing, which is typically used in e.g. maize, is the mechanical elimination of all male flowers (detasseling). The only flowers remaining on the plant are female, and these can be manually pollinated with pollen from a selected paternal line, in order to obtain ears with exclusively hybrid kernels.

In plant species with hermaphroditic flowers (producing both ovules and pollen grains within the same flower), a common strategy for preventing selfing is emasculation by mechanical removal of anthers and/or pollen prior to anthesis. When the anthers are mechanically removed before the pollen grains are released from the loculi and/or before the filament has extended far enough to match the height of the stigma, selfing is efficiently prevented. Subsequently the female reproductive parts of the emasculated flower are allowed to mature normally, after which pollen grains from a selected father plant can be deposited on the stigma, in order to obtain exclusively hybrid seeds from the cross. Especially for commercial-scale applications this method is however very labour-intensive and not 100% reliable: if anthers are removed in a slightly too late developmental stage or if one anther is accidentally not removed, this can lead to a mixed seed set, consisting of hybrid and maternal seeds. This results in non-uniformity of the commercial seed batch, which is undesired for customers who expect uniform and consistently superior seeds, and it brings inbred mother lines of hybrid varieties into commerce, which is undesired for the breeding company. A 100% reliable hybrid system is therefore desirable.

Male sterility in plants occurs when the anthers or pollen of the plant are non-functional. This condition is manifested as an extreme reduction or absence of pollen, the malformation or absence of flowers or stamens, or the failure of pollen to dehisce. The induction of male sterility can occur by means of chemicals. This so-called male gametocide can be achieved by treatment with e.g. gibberellins (in rice and maize), sodium methyl arsenate (in rice), or maleic acid (in wheat and onion). Disadvantages of this approach are the fact that this male sterility is not inheritable as it does not result from a genetic determinant present in the plant's genome, and that chemical treatment is labour intensive and not 100% reliable.

Another category of mechanisms through which selfing can be prevented, is termed genetic male sterility. Here three different approaches can be distinguished: genetic-engineered male sterility (transgenic MS), cytoplasmic male sterility (CMS) and genic male sterility (GMS). Transgenic MS comprises all approaches that use a transgene to ensure that pollen grains are unable to fertilise ovules, that either lead to the death of pollen grains prior to anthesis, or to the dysfunctionality of pollen grains at anthesis. A well-known example is the reversible Barnase/Barstar system, wherein the Barnase enzyme is transgenically expressed in the tapetum, which leads to pollen sterility. However, when the Barstar protein is co-expressed in the tapetum, it blocks Barnase activity and restores pollen fertility.

CMS is a type of sterility that is under control of extra-nuclear, cytoplasmic factors, more precisely of plastid origin. Usually, mutations in the mitochondrial genome underlie CMS, and they typically inherit in a maternal fashion. Routine hybrid seed production with CMS lines requires the use of maintainer and restorer lines, which complicates the process and increases the costs and time required for commercial hybrid seed production.

GMS encompasses a nuclear influence on male fertility, in contrast to cytoplasmic influences which are caused by organellar factors. Due to e.g. a mutation in a nuclear gene the plant does not produce viable and/or functional pollen grains or male spores, and/or it is unable to disperse its pollen due to e.g. non-dehiscence of its anthers.

Watermelon belongs to the genus Citrullus which is part of the Cucurbit family (Cucurbitaceae). The modern cultivated watermelon is known as Citrullus lanatus var. lanatus (Thunb.) Matsum. & Nakai. Watermelon is grown throughout the tropical and sub-tropical regions of the world, predominantly for consumption of its sweet flesh. The Southern part of the USA, China, the Middle East, Africa, India, Japan and Southern Europe are the most important watermelon producing areas.

Watermelon is a monoecious species, whereby both male and female flowers are present on the same plant. For hybrid watermelon production of highly pure seeds, controlled pollination is necessary. Controlled pollination requires hand pollination of the female flowers of the female parent with pollen of a selected paternal line. This method prevents undesirable self-pollination from the male flowers of the female parent or unwanted cross pollination, but it is time consuming and labour intensive, all of which, notably increases production costs.

Given the current practice, it is an object of the present invention to provide a means of addressing this costly and laborious system, by eliminating the need for controlled pollination in watermelon hybrid production.

Citation or identification of any document in this application is not an admission that such document is available as prior art to the present invention.

### SUMMARY OF THE INVENTION

In one aspect, the present invention relates to a watermelon (Citrullus lanatus) plant comprising a modified Male Meiocyte Death 1-like (MMD1-like) gene;
wherein SEQ ID NO: 2 is the nucleotide sequence of the wild type MMD1-like gene;
wherein the modified MMD1-like gene encodes a MMD1-like protein comprising a modification in the wild type amino acid sequence of SEQ ID NO. 1, or in an amino acid sequence having at least 95% sequence identity to SEQ ID NO. 1;
wherein:
(a) the modified MMD1-like gene comprises a premature stop codon encoding and resulting in expression of the modified MMD1-like protein lacking the PHD_MMD1_like domain;
   wherein the PHD_MMD1_like domain is amino acids at position 551 to 595 of SEQ ID NO: 1;
(b) the modified MMD1-like gene comprises a substitution of a guanine with an adenine at position 12 of SEQ ID NO: 2;
(c) the modified MMD1-like gene has the sequence of SEQ ID NO: 4; or
(d) the modified MMD1-like protein has the amino acid sequence of SEQ ID NO: 3; and
the modified MMD1-like gene, when homozygously present, confers genic male sterility to the plant.

In some embodiments, the amino acid sequence encoded by the modified MMD1-like gene has at least 99% sequence identity to SEQ ID NO. 1.

In some embodiments, the modified MMD1-like gene comprises the premature stop codon encoding and resulting in expression of the modified MMD1-like protein lacking the PHD_MMD1 like domain.

In some embodiments, the premature stop codon is the result of a nucleotide substitution from guanine to adenine at position 12 of SEQ ID NO. 2.

In some embodiments, the modified MMD1-like gene has the sequence of SEQ ID NO: 4.

In some embodiments, the MMD1-like protein encoded by the modified MMD1-like gene has the amino acid sequence of SEQ ID NO. 3.

In some embodiments, the modified MMD1-like gene is homozygously present and confers genic male sterility to the plant.

In some embodiments, the modified MMD1-like gene is heterozygously present.

In some embodiments, the modified MMD1-like gene encoding the modified MMD-1 protein when homozygously present in the genome of the plant, confers genic male sterility.

In another aspect, the present invention relates to a watermelon (Citrullus lanatus) seed from, or that produces, the watermelon (Citrullus lanatus) plant comprising the modified MMD1-like gene as described herein.

In another aspect, the present invention relates to a progeny plant of the watermelon (Citrullus lanatus) plant as described herein, wherein the progeny plant comprises the modified MMD1-like gene.

In another aspect, the present invention relates to a fruit harvested from the watermelon (Citrullus lanatus) plant as described herein, wherein the fruit comprises the modified MMD1-like gene.

In another aspect, the present invention relates to a propagation material capable of developing into, and/or having been derived from, the watermelon (Citrullus lanatus) plant as described herein, wherein the propagation material comprises the modified MMD1-like gene, and wherein the propagation material comprises a microspore, a pollen, an ovary, an ovule, an embryo, an embryo sac, an egg cell, a cutting, a root, a root tip, a hypocotyl, a cotyledon, a stem, a leave, a flower, an anther, a seed, a meristematic cell, a protoplast, or a cell or a tissue culture thereof.

In yet another aspect, the present invention relates to a method for producing a watermelon (Citrullus lanatus) plant that exhibits genic male sterility comprising introducing into the genome of a watermelon (Citrullus lanatus) plant a modified Male Meiocyte Death 1-like (MMD1-like) gene;
wherein SEQ ID NO: 2 is the nucleotide sequence of the wild type MMD1-like gene;
wherein the modified MMD1-like gene encodes a MMD1-like protein comprising a modification in the wild type amino acid sequence of SEQ ID NO. 1, or in an amino acid sequence having at least 95% sequence identity to SEQ ID NO. 1;
wherein:
(a) the modified MMD1-like gene comprises a premature stop codon encoding and resulting in expression of the modified MMD1-like protein lacking the PHD_MMD1_like domain;
   wherein the PHD_MMD1_like domain is amino acids at position 551 to 595 of SEQ ID NO: 1;
(b) the modified MMD1-like gene comprises a substitution of a guanine with an adenine at position 12 of SEQ ID NO: 2;
(c) the modified MMD1-like gene has the sequence of SEQ ID NO: 4; or
(d) the modified MMD1-like protein has the amino acid sequence of SEQ ID NO: 3; and
the modified MMD1-like gene, when homozygously present, confers genic male sterility to the plant.

In some embodiments, the modified MMD1-like gene is introduced into the genome of the watermelon (Citrullus lanatus) plant by means of mutagenesis, introgression, cis-genesis, transgenesis, or combinations thereof.

In another aspect, the present invention relates to a marker for the detection of a modified Male Meiocyte Death 1-like (MMD1-like) gene;
wherein SEQ ID NO: 2 is the nucleotide sequence of the wild type MMD1-like gene;
wherein the modified MMD1-like gene encodes a MMD1-like protein comprising a modification in the wild type amino acid sequence of SEQ ID NO. 1, or in an amino acid sequence having at least 95% sequence identity to SEQ ID NO. 1;
wherein the modified MMD1-like gene comprises a substitution of a guanine with an adenine at position 12 of SEQ ID NO: 2; and
the marker comprises an oligonucleotide that detects the nucleotide substitution of the guanine with the adenine at position 12 of SEQ ID NO. 2.

In some embodiments, the marker comprises the sequence of SEQ ID NO. 5 or SEQ ID NO. 6, or parts thereof.

In another aspect, the present invention relates to a method of selecting a watermelon (Citrullus lanatus) plant that exhibits genic male sterility comprising identifying the presence of a modified Male Meiocyte Death 1-like (MMD1-like) gene; and selecting a plant that homozygously comprises said modified MMD1-like gene as a plant that exhibits genic male sterility;
wherein SEQ ID NO: 2 is the nucleotide sequence of the wild type MMD1-like gene;
wherein the modified MMD1-like gene encodes a MMD1-like protein comprising a modification in the wild type amino acid sequence of SEQ ID NO. 1, or in an amino acid sequence having at least 95% sequence identity to SEQ ID NO. 1;
wherein:
(a) the modified MMD1-like gene comprises a premature stop codon encoding and resulting in expression of the modified MMD1-like protein lacking the PHD_MMD1_like domain;
   wherein the PHD_MMD1_like domain is amino acids at position 551 to 595 of SEQ ID NO: 1;
(b) the modified MMD1-like gene comprises a substitution of a guanine with an adenine at position 12 of SEQ ID NO: 2;
(c) the modified MMD1-like gene has the sequence of SEQ ID NO: 4; or
(d) the modified MMD1-like protein has the amino acid sequence of SEQ ID NO: 3; and
the modified MMD1-like gene, when homozygously present, confers genic male sterility to the plant.

In some embodiments, the identifying is performed with a marker that detects the modified MMD1-like gene having the substitution of the guanine with the adenine at position 12 of SEQ ID NO: 2. In some embodiments, the marker is as described herein.

In another aspect, the present invention relates to a method of selecting a watermelon (Citrullus lanatus) plant comprising a modified Male Meiocyte Death 1-like (MMD1-like) gene, comprising:
a) identifying by genetic analysis the presence of the modified MMD1-like gene in the genome of the watermelon (Citrullus lanatus) plant;
b) selecting a plant that comprises the modified MMD1-like gene;
c) optionally further testing the selected plant for genic male sterility as compared to an isogenic watermelon (Citrullus lanatus) plant lacking the modified MMD1-like gene, and
d) optionally selecting the further tested plant exhibiting genic male sterility as compared to the isogenic watermelon (Citrullus lanatus) plant lacking the modified MMD1-like gene;
   wherein SEQ ID NO: 2 is the nucleotide sequence of the wild type MMD1-like gene;
   wherein the modified MMD1-like gene encodes a MMD1-like protein comprising a modification in the wild type amino acid sequence of SEQ ID NO. 1, or in an amino acid sequence having at least 95% sequence identity to SEQ ID NO. 1;
   wherein:
   (i) the modified MMD1-like gene comprises a premature stop codon encoding and resulting in expression of the modified MMD1-like protein lacking the PHD_MMD1_like domain;
      wherein the PHD_MMD1_like domain is amino acids at position 551 to 595 of SEQ ID NO: 1;
   (ii) the modified MMD1-like gene comprises a substitution of a guanine with an adenine at position 12 of SEQ ID NO: 2;
   (iii) the modified MMD1-like gene has the sequence of SEQ ID NO: 4; or
   (iv) the modified MMD1-like protein has the amino acid sequence of SEQ ID NO: 3; and
   the modified MMD1-like gene, when homozygously present, confers genic male sterility to the plant.

In some embodiments, the method of selecting further comprises the steps of:
c) further testing the selected plant for genic male sterility as compared to an isogenic watermelon (Citrullus lanatus) plant lacking the modified MMD1-like gene, and
d) selecting the further tested plant exhibiting genic male sterility as compared to the isogenic watermelon (Citrullus lanatus) plant lacking the modified MMD1-like gene.

In yet another aspect, the present invention relates to a method for producing a watermelon (Citrullus lanatus) plant, comprising the steps of:
(a) crossing a first watermelon parent plant whose genome comprises a modified Male Meiocyte Death 1-like (MMD1-like) gene with a second watermelon (Citrullus lanatus) parent plant to obtain an F1 population;
(b) optionally performing one or more rounds of selfing and/or crossing with a F1 watermelon (Citrullus lanatus) plant to obtain a further generation population;
(c) selecting from the population resulting from the cross of step a) or from the further generation population of step b), a watermelon (Citrullus lanatus) plant that comprises the modified MMD1-like gene;
wherein SEQ ID NO: 2 is the nucleotide sequence of the wild type MMD1-like gene;
wherein the modified MMD1-like gene encodes a MMD1-like protein comprising a modification in the wild type amino acid sequence of SEQ ID NO. 1, or in an amino acid sequence having at least 95% sequence identity to SEQ ID NO. 1;
wherein:
(i) the modified MMD1-like gene comprises a premature stop codon encoding and resulting in expression of the modified MMD1-like protein lacking the PHD_MMD1_like domain;
   wherein the PHD_MMD1_like domain is amino acids at position 551 to 595 of SEQ ID NO: 1;
(ii) the modified MMD1-like gene comprises a substitution of a guanine with an adenine at position 12 of SEQ ID NO: 2;
(iii) the modified MMD1-like gene has the sequence of SEQ ID NO: 4; or
(iv) the modified MMD1-like protein has the amino acid sequence of SEQ ID NO: 3; and
the modified MMD1-like gene, when homozygously present, confers genic male sterility to the plant.

In yet another aspect, the present invention relates to a method for the production of hybrid watermelon (Citrullus lanatus) seed, comprising:
crossing a first watermelon (Citrullus lanatus) parent plant with a second watermelon (Citrullus lanatus) parent plant and harvesting the resultant hybrid seed;
wherein the first watermelon (Citrullus lanatus) parent plant or the second watermelon parent plant comprises a modified Male Meiocyte Death 1-like (MMD1-like) gene;
wherein SEQ ID NO: 2 is the nucleotide sequence of the wild type MMD1-like gene;
wherein the modified MMD1-like gene encodes a MMD1-like protein comprising a modification in the wild type amino acid sequence of SEQ ID NO. 1, or in an amino acid sequence having at least 95% sequence identity to SEQ ID NO. 1;
wherein:
(i) the modified MMD1-like gene comprises a premature stop codon encoding and resulting in expression of the modified MMD1-like protein lacking the PHD_MMD1_like domain;
   wherein the PHD_MMD1_like domain is amino acids at position 551 to 595 of SEQ ID NO: 1;
(ii) the modified MMD1-like gene comprises a substitution of a guanine with an adenine at position 12 of SEQ ID NO: 2;
(iii) the modified MMD1-like gene has the sequence of SEQ ID NO: 4; or
(iv) the modified MMD1-like protein has the amino acid sequence of SEQ ID NO: 3; and the modified MMD1-like gene, when homozygously present, confers genic male sterility to the plant.

In some embodiments, the second watermelon (Citrullus lanatus) parent plant comprises the modified MMD1-like gene.

In yet another aspect, the present invention relates to a hybrid watermelon (Citrullus lanatus) seed produced by the method described herein.

In yet another aspect, the present invention relates to an isolated non-naturally occurring nucleic acid molecule encoding a MMD1-like protein comprising a modification in the wild type amino acid sequence of SEQ ID NO. 1, or in an amino acid sequence having at least 95% sequence identity to SEQ ID NO. 1;
wherein SEQ ID NO: 2 is the nucleotide sequence of the wild type nucleic acid molecule;
wherein:
(a) the isolated non-naturally occurring nucleic acid molecule comprises a premature stop codon encoding and resulting in expression of the modified MMD1-like protein lacking the PHD_MMD1_like domain;
   wherein the PHD_MMD1_like domain is amino acids at position 551 to 595 of SEQ ID NO: 1;
(b) the isolated non-naturally occurring nucleic acid molecule comprises a substitution of a guanine with an adenine at position 12 of SEQ ID NO: 2;
(c) the isolated non-naturally occurring nucleic acid molecule has the sequence of SEQ ID NO: 4; or
(d) the modified MMD1-like protein encoded by the isolated non-naturally occurring nucleic acid molecule has the amino acid sequence of SEQ ID NO: 3.

In some embodiments, the isolated non-naturally occurring nucleic acid molecule comprises the premature stop codon encoding and resulting in expression of the modified MMD1-like protein lacking the PHD_MMD1_like domain.

In some embodiments, the isolated non-naturally occurring nucleic acid molecule comprises the substitution of the guanine with the adenine at position 12 of SEQ ID NO: 2.

In some embodiments, the isolated non-naturally occurring nucleic acid molecule comprises the sequence of SEQ ID NO: 4.

In some embodiments, the modified MMD1-like protein encoded by the isolated non-naturally occurring nucleic acid molecule as described herein has the amino acid sequence of SEQ ID NO: 3.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112(a)) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

FIG. 1A shows the sequences of the wild type MMD-1 like protein sequence (SEQ ID NO. 1) and wild type MMD1-like gene CDS (SEQ ID NO. 2); Mutant MMD-1 like protein sequence (SEQ ID NO. 3) and mutant MMD1-like gene CDS (SEQ ID NO. 4). The underlined and bolded amino acid or nucleotide depicts the position of the respective amino acid or nucleotide that is affected by the modification. * denotes the truncation of the protein due to a premature stop codon in the corresponding nucleotide sequence.

FIG. 1B shows nucleotide sequences of the MMD1-like gene to be used for developing markers (SEQ ID NO. 5 and SEQ ID NO. 6). The Haplotype of the wild type and mutant alleles are denoted. The underlined and bolded nucleotide depicts the position of the respective nucleotide that is affected by the modification.

FIG. 2 shows a representative photograph of a male flower (A), anthers from a male flower (B) and lack of viable pollen grains of the male flower (C), obtained from plants comprising a homozygously mutant MMD1-like gene; Representative photograph of a male flower (D), anthers from a male flower (E) and presence of viable pollen grains of the male flower (F), obtained from plants comprising a wild type MMD1-like gene.

FIG. 3 is a scatter plot showing the genetic relationship of F1 progeny seeds in relation to their GMS mother and Non-GMS father line.

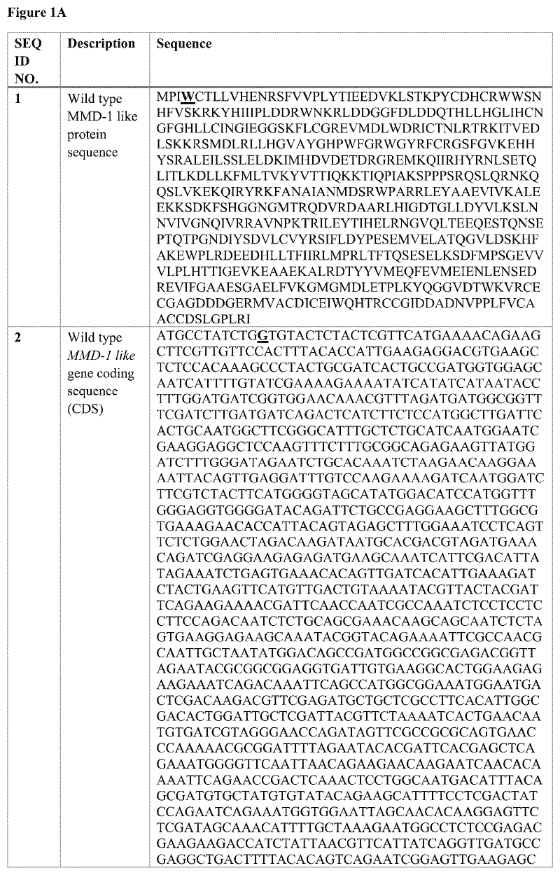

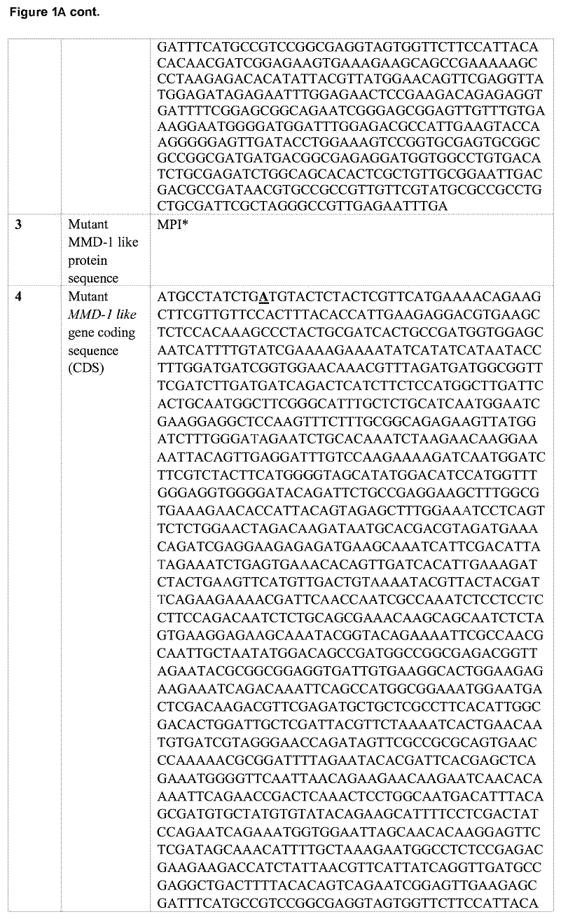

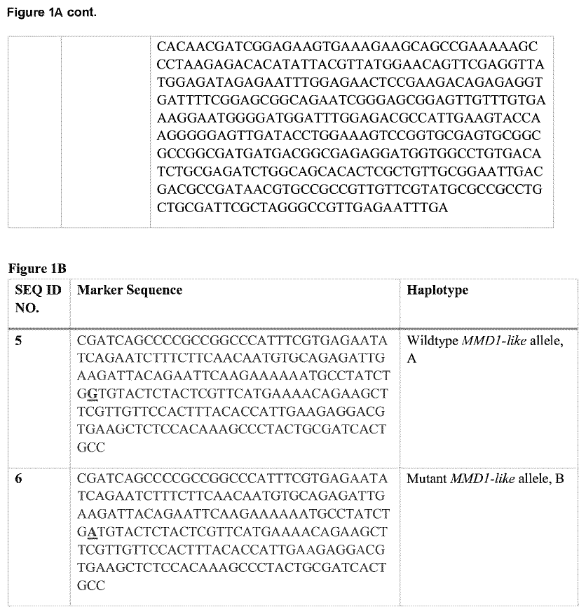

The following detailed description, given by way of example, but not intended to limit the invention solely to the specific embodiments described, may best be understood in conjunction with the accompanying drawings.

### DETAILED DESCRIPTION OF THE INVENTION

In the research leading to the present invention, novel genic male sterile watermelon plants were developed. A genic male sterile watermelon plant comprises flowers with non-viable or absent pollen or pollen producing flowers are eliminated altogether, thereby producing an ideal female parent. Hand pollination can be eliminated from the production process if using male sterile plants. Instead, insect pollination can be used when male sterile (female) plants and the desired male fertile plants are planted together. The GMS trait was surprisingly found to have resulted from a modification of the Male Meiocyte Death 1-like (MMD1-like) gene. MMD1-like encodes a protein comprising a domain which belongs to the conserved protein domain family, PHD_MMD1_like (CDD entry: cd15556). Proteins belonging to the PHD_MMD1_like domain family are plant homeodomain (PHD) zinc finger proteins, which are expressed in male meiosis. PHD zinc fingers are often found in transcriptional regulatory proteins and proteins associated with chromatin remodelling complexes, suggesting that the MMD1-like protein could act as a transcriptional regulator in male meiocytes.

The GMS trait of the invention is controlled by modification(s) to the MMD1-like gene, the inheritance of which is consistent with that of a monogenic recessive trait. The term “recessive trait” is to mean in the context of this application that the fully achievable trait is observed when the modified MMD1-like gene is homozygously present in the genome such that both alleles of the MMD1-like gene comprise the modification. When the modified MMD1-like gene is heterozygously present in the genome, only one allele of the MMD1-like gene is modified and therefore does not confer GMS. Since the inheritance of the trait is comparable to that of a monogenic trait, it is advantageous in that the trait can easily be incorporated into various plant types for a given plant species.

A “gene” in the context of this application comprises exonic sequences and regulatory sequences such as a promotor sequence, and optionally intronic sequences. In this application the term “modification” or “modified” refers to a change in the sequence of the wild type MMD1-like gene that results in an altered version of the wild type gene. A change or modification to the coding sequence (CDS) of the gene leads to a change in the amino acid sequence of the encoded protein, whereas a change or modification to the regulatory sequences of the gene in turn leads to a change in the transcription of the gene. The resultant modified MMD1-like protein has a reduced level, reduced activity or complete absence of the encoded MMD1-like protein. As used herein, “wild type” refers to the typical or most common, non-mutated or unmodified form of an organism, strain, gene, protein, characteristic or trait and is in contrast to a mutated or modified form for example. In the context of the invention, the wild type MMD1-like gene does not confer GMS.

The modified MMD1-like gene of the invention encodes a modified protein comprising one or more modifications in the wild type amino acid sequence of SEQ ID NO. 1. In another aspect of the invention, the modified MMD1-like gene of the invention encodes a modified protein comprising one or more modifications in an amino acid sequence having at least 90% sequence identity to SEQ ID NO. 1, preferably at least 91%, 92%, 93%, 94%, 95%, 96%, 97%, 98%, or 99% sequence identity to SEQ ID NO. 1. The modification is not included in calculating the difference in sequence identity since the modification should always be present. The skilled person is familiar with methods for calculating sequence identity. Suitably sequence identity is calculated using the Sequence Identities and Similarities (SIAS) tool, which can be accessed at imed.med.ucm.es/Tools/sias.html. SIAS calculates pairwise sequence identity and similarity percentages between each pair of sequences from a multiple sequence alignment. Sequence identity is calculated using a method taking the gaps into account; sequence similarity is calculated based on grouping of amino acids having similar properties. For calculations, default settings for SIM percentage, similarity amino acid grouping, sequence length, normalized similarity score, matrix and gap penalties are used.

The invention thus relates to a modified MMD1-like gene, the wild type of which encodes a protein that comprises the amino acid sequence as identified in SEQ ID NO. 1.

The wild type of the MMD1-like gene of this invention comprises the nucleotide sequence of SEQ ID NO: 2. In the publicly available genome assembly of Citrullus lanatus cv. 97103 (version 1, see Guo et al, 2013, the draft genome of watermelon (Citrullus lanatus) and resequencing of 20 diverse accessions. Nature Genetics 45(1):51-58) said wild type of the modified MMD1-like gene of this invention is located on chromosome 10 at position 1185039-1187019 (+). Also encompassed by the term “wild type of the MMD1-like gene of this invention” is a gene having a nucleotide sequence that has, in order of increased preference, at least 90%, 91%, 92%, 93%, 94%, 95%, 96%, 97%, 98% or 99% sequence identity to the nucleotide sequence of SEQ ID NO:2.

The wild type of the MMD1-like gene of the invention encodes the protein comprising the amino acid sequence of SEQ ID NO: 1. This wild type MMD1-like protein comprises the following conserved domain: PHD_MMD1_like domain (aa 551-595 of SEQ ID NO: 1). Also encompassed by the term “wild type MMD1 gene of the invention” is a gene that encodes a protein having an amino acid sequence that has, in order of increased preference, at least 90%, 91%, 92%, 93%, 94%, 95%, 96%, 97%, 98% or 99% sequence identity to the amino acid sequence of SEQ ID NO: 1.

In one embodiment the modified MMD1-like gene of the invention is a nucleic acid, in particular a nucleic acid molecule, more in particular an isolated nucleic acid molecule.

The DNA sequence of a gene may be altered in a number of ways, and will have varying effects depending on where the modification(s) occur and whether they alter the function of the encoded protein. Examples of such modifications include amino acid substitutions, premature stop codons, insertions, deletions, or frameshift mutations.

An insertion changes the number of DNA bases in a gene by adding one or more base pairs. A deletion changes the number of DNA bases by removing one or a few base pairs, or even an entire gene or neighboring genes. These types of modifications may alter the function of the encoded protein.

Frame shift mutations are caused by insertion or deletion of one or more base pairs in a DNA sequence encoding a protein. When the number of inserted or deleted base pairs at a certain position is not a multiple of 3, the triplet codon encoding the individual amino acids of the protein sequence become shifted relative to the original open reading frame, and then the encoded protein sequence changes dramatically. Protein translation will result in an different amino acid sequence than that of the originally encoded protein, and often a frameshift can lead to a premature stop codon in the open reading frame. The overall result is that the encoded protein no longer has the same biological function as the originally encoded protein.

An amino acid substitution in an encoded protein sequence arises when the mutation of one or more base pairs in the coding sequence results in an altered triplet codon, often encoding a different amino acid. Due to the redundancy of the genetic code not all point mutations lead to amino acid changes. Such mutations are termed “silent mutations”. Some amino acid changes are “conservative”, i.e. they lead to the replacement of one amino acid by another amino acid with comparable properties, such that the mutation is unlikely to dramatically change the folding of the mature protein, or influence its function. Conservative amino acid substitutions may be made on the basis of chemical properties, for example similarity in polarity, charge, solubility, hydrophobicity, hydrophilicity or amphipathic nature of the residues, in which case the resulting protein may still function normally. Other amino acid changes are non-silent, non-conservative amino acid changes in domains that play a role in substrate recognition, the active site of enzymes, interaction domains or in major structural domains (such as transmembrane helices) may partly or completely destroy the functionality of an encoded protein, without thereby necessarily affecting the expression level of the encoding gene. Whether an amino acid substitution is conservative or non-conservative may be predicted on the basis of chemical properties, for example similarity in polarity, charge, solubility, hydrophobicity, hydrophilicity or amphipathic nature of the amino acids.

A deletion, insertion, frame shift mutation and/or amino acid substitution may result in a nonsense mutation. A nonsense mutation is a mutation in a nucleic acid molecule encoding a protein whereby a codon is changed into a premature stop codon. Converting an amino acid into a premature stop codon results in a truncated protein. How much of the protein is lost determines whether or not the protein is still functional. Especially when all or part of the conserved functional domains are lacking from the truncated protein it is likely protein function is affected. Premature stop codons may also lead to nonsense-mediated decay, in which mRNAs that are transcribed from an allele carrying a nonsense mutation are eliminated, leading to low RNA expression levels and no or very little protein.

A deletion, insertion, frame shift mutation and/or amino acid substitution may result in a null mutation or knockout mutation. A null mutation or knockout mutation is a mutation that eliminates the function of the affected gene. For example, a null mutation in a gene that usually encodes a specific enzyme leads to the production of a nonfunctional enzyme or no enzyme at all.

The wild type of the MMD1-like gene of the invention encodes a protein comprising the amino acid sequence of SEQ ID NO: 1. The wild type MMD1-like protein comprises the following conserved domain: PHD_MMD1-like domain (aa 551-595 of SEQ ID NO: 1). Also encompassed by the term “wild type of the MMD1-like gene of this invention” is a gene that encodes a protein having an amino acid sequence that has, in order of increased preference, at least 90%, 91%, 92%, 93%, 94%, 95%, 96%, 97%, 98% or 99% sequence identity to the amino acid sequence of SEQ ID NO: 1.

The modified MMD1-like gene of the invention comprises one or more nucleotides that are replaced, inserted and/or deleted relative to the wild type gene sequence, and said one or more replaced, inserted and/or deleted nucleotides result in an absence of functional MMD1-like protein.

In the context of this invention the term “absence of functional MMD1-like protein” means that either no MMD1-like protein is expressed, or that the MMD1-like protein expressed is non-functional and does not have MMD1-like activity. The modification to the MMD1-like gene can lead to the absence of MMD-1 like RNA or a significantly decreased MMD-1 like RNA level, resulting in an absence of MMD1-like protein. Alternatively, the modified MMD-1 like protein is expressed but is non-functional: an absence of one or more of the functional domains of the MMD-1 like protein results in a modified MMD1-like protein that cannot perform its function in transcription regulation in male meiocytes.

In one embodiment, the modified MMD1-like gene of the invention comprises a premature stop codon that leads to an absence of functional MMD1-like protein. In another embodiment, the modified MMD1-like gene of the invention comprises a premature stop codon resulting in the absence of the PHD_MMD1_like domain from the encoded modified MMD1-like protein. In a preferred embodiment, the one or more nucleotides that are replaced, inserted and/or deleted in the modified MMD1-like gene of the invention relative to the wild type are at position 1 to 1815 of SEQ ID NO: 2, resulting in a premature stop codon that leads to an absence of functional protein. In a most preferred embodiment, the modified MMD1-like gene comprises a replacement of a guanine to adenine at position 12 (i.e. G12A) of SEQ ID NO: 2.

The modified MMD1-like gene of this invention confers GMS to a plant when homozygously present.

In one embodiment, the modified MMD1-like gene of the invention is a nucleic acid, in particular a nucleic acid molecule, and more in particular an isolated nucleic acid molecule.

The invention relates to a watermelon plant comprising the modified MMD1-like gene of the invention, wherein the homozygous presence of the modified MMD1-like gene confers GMS to the plant. The plant can comprise the modified MMD1-like gene of the invention heterozygously, in which case the plant is not genic male sterile but the plant is useful for transferring the modified MMD1-like gene of the invention to another plant.

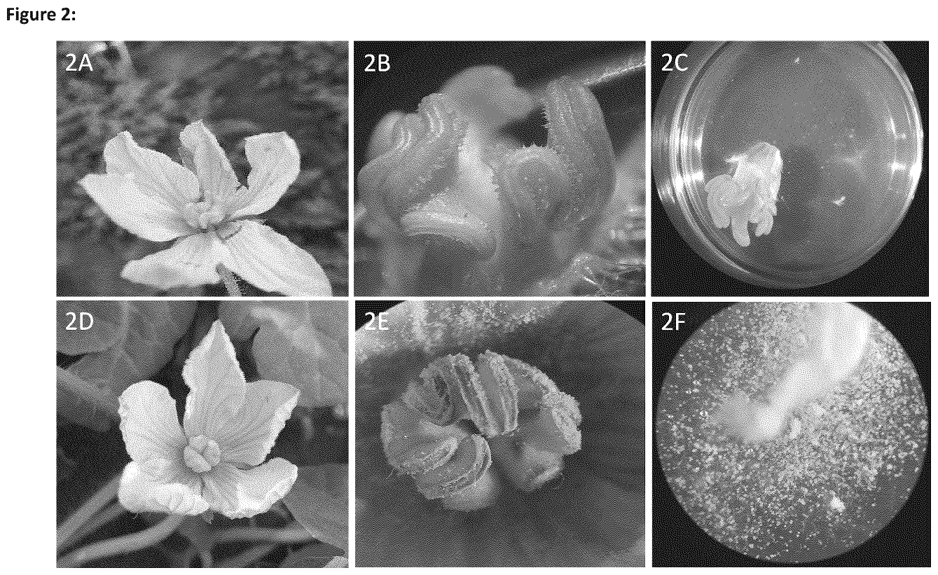

Genic male sterile watermelon plants comprise both male and female flowers. The female flowers of a genic male sterile watermelon plant have a normal flower morphology. The male flowers can either have a normal flower morphology, or the male flowers can be aborted. The major difference between the male flowers of a genic male sterile watermelon plant compared to the male flowers of a wild type (fertile) watermelon plant, is that the male flowers of genic male sterile plants either have non-viable pollen grains or do not produce pollen all together (See FIGS. 2A, 2B, 2C as compared to FIG. 2D, 2E, 2F). As described herein, the lack of viable pollen grains is attributed to the modified MMD1-like gene of this invention.

As used herein the term “watermelon plant of the invention” or “plant of the invention” is intended to refer to a watermelon (Citrullus lanatus var. lanatus) plant comprising the modified MMD1-like gene of the invention.

The watermelon plant of the invention can be a watermelon plant of any type, any fruit form or fruit color, and is preferably an agronomically elite watermelon plant. In the context of this invention, an agronomically elite plant is a plant having a genotype that, as a result of directed crossing and selection by human interventions, comprises an accumulation of distinguishable and desirable agronomic traits which allow a producer to harvest a product of commercial significance.

In one embodiment, the mature fruits of the watermelon plant of the invention have red, orange or yellow flesh. In another embodiment, the mature fruits of said plant have flesh with soluble solids of at least, in order of increased preference, 5.0 degrees Brix, 6.0 degrees Brix, 7.0 degrees Brix, 8.0 degrees Brix, 9.0 degrees Brix, 9.5 degrees Brix, 10.0 degrees Brix, 10.5 degrees Brix, 11.0 degrees Brix, 11.5 degrees Brix, 12.0 degrees Brix, 12.5 degrees Brix, 13.0 degrees Brix, 13.5 degrees Brix, 14.0 degrees Brix, 14.5 degrees Brix, 15.0 degrees Brix, 15.5 degrees Brix, 16.0 degrees Brix, or 17.0 degrees Brix. The soluble solids of the mature fruits of said plant are suitably not higher than 18 degrees Brix. In another embodiment the watermelon plant of the invention is a plant of an inbred line or a hybrid plant.

In this application all watermelon plants referred to are diploid. If a plant referred to in this application is not diploid it will be indicated that it is triploid or tetraploid. The plant of the invention may be a diploid, a tetraploid or a triploid watermelon plant. A triploid watermelon plant of the invention can be produced by treating a diploid watermelon line comprising the modified MMD1-like gene heterozygously with colchicine to obtain a tetraploid watermelon plant which after successive selfings comprises the MMD1-like gene homozygously and is genic male sterile. The genic male sterile tetraploid plant can successively be crossed with a diploid watermelon plant not comprising the modified MMD1-like gene and therefore is not genic male sterile, to generate triploid watermelon plants that produce triploid seedless watermelon fruit.

The plant of the invention may be a plant of an inbred line, a hybrid, a doubled haploid, or a plant of a segregating population. As used herein, a plant of an inbred line is a plant of a population of plants that is the results of three or more rounds of selfing, or backcrossing; or which plant is double haploid. An inbred line may for example be a parent line used for the production of a commercial hybrid.

As used herein, a “hybrid plant” is a plant which is the result of a cross between two different plants having different genotypes. More in particular, a hybrid plant is the result of a cross between plants of two different inbred lines, such that a hybrid plant may e.g. be a plant of an F₁ hybrid variety.

The invention also encompasses a watermelon seed, comprising the modified MMD1-like gene of the invention, wherein the plant grown from said seed is genic male sterile as a result of the homozygous presence of the modified MMD1-like gene.

The invention further relates to a part of the watermelon plant of the invention, which comprises a fruit of the plant of the invention or a seed of the plant of the invention, wherein the plant part comprises the modified MMD1-like gene of the invention.

The invention further relates to a watermelon fruit produced by the watermelon plant of the invention. This watermelon fruit is a fruit of the invention.

Moreover, the invention also relates to a food product or a processed food product comprising the fruit of the invention or a part thereof. The food product may have undergone one or more processing steps. Such a processing step might comprise but is not limited to any one of the following treatments or combinations thereof: peeling, cutting, washing, juicing, cooking, cooling or preparing a salad mixture comprising the fruit of the invention. The processed form that is obtained is also part of this invention since it comprises DNA in which the modified MMD1-like is present.

The invention further relates to a cell of a plant of the invention, which cell comprises a modified MMD1-like gene of the invention. Such a cell may either be in isolated form or a part of the complete plant or parts thereof and still constitutes a cell of the invention because such a cell harbors the genetic information that imparts GMS. Each cell of a plant of the invention carries the genetic information that leads to GMS. A cell of the invention may also be a regenerable cell that can regenerate into a new plant of the invention. The presence of genetic information as used herein is the presence of the modified MMD1-like gene of the invention.

The invention further relates to plant tissue of a plant of the invention, which tissue comprises the modified MMD1-like gene of the invention. The tissue can be undifferentiated tissue or already differentiated tissue. Undifferentiated tissue is for example a stem tip, an anther, a petal, or pollen, and can be used in micropropagation to obtain new plantlets that are grown into new plants of the invention. The tissue can also be grown from a cell of the invention.

The invention moreover relates to progeny of a plant, a cell, a tissue, or a seed of the invention, which progeny comprises the modified MMD1-like gene of the invention. Such progeny can in itself be a plant, a cell, a tissue, or a seed. As used herein “progeny” is intended to mean the first and all further descendants from a cross with a plant of the invention, wherein a cross comprises a cross with itself or a cross with another plant, and wherein a descendant that is determined to be progeny comprises the modified MMD1-like gene of the invention. Progeny also encompasses material that is obtained by vegetative propagation or another form of multiplication.

The parent can also be a progeny plant from the seed, or a progeny plant from seeds that are identified to have (or to have acquired) the trait of the invention by other means. In one embodiment, the invention relates to watermelon (Citrullus lanatus var. lanatus) plants that carry the trait of the invention and that have acquired the said trait by introduction of the genetic information that is responsible for the trait from a suitable source, either by conventional breeding, or genetic modification, in particular by cis-genesis or transgenesis. Cis-genesis is genetic modification of plants with a natural gene, encoding an (agricultural) trait from the crop plant itself or from a sexually compatible donor plant. Trans-genesis is genetic modification of a plant with a gene from a non-crossable species or with a synthetic gene. The invention also relates to propagation material capable of developing into or being derived from a plant of the invention, wherein the propagation material comprises the modified MMD1-like gene of the invention. The propagation material is selected from a group consisting of a microspore, a pollen, an ovary, an ovule, an embryo, an embryo sac, an egg cell, a cutting, a root, a root tip, a hypocotyl, a cotyledon, a stem, a leave, a flower, an anther, a seed, a meristematic cell, a protoplast and a cell, or a tissue culture thereof.

The invention further relates to use of the modified MMD1-like gene of the invention for producing a plant that is genic male sterile. The genic male sterile plant may be produced by introduction of the modified MMD1-like gene into its genome, in particular by means of mutagenesis, introgression, cis-genesis or transgenesis or combinations thereof.

The invention further relates to a marker for the identification of a modified MMD1-like gene, wherein the marker comprises any of the modifications in a MMD1-like gene as described herein and can thereby identify said modifications. Such marker for identification comprises a nucleotide sequence that includes a particular polymorphism in its sequence, when compared to the same sequence stretch in a wild type MMD1-like gene, which polymorphism leads to a modification in the encoded protein sequence that changes the function or activity of the MMD1-like protein. A marker of the invention is in particular a marker comprising an oligonucleotide that detects a single nucleotide polymorphism (SNP) from guanine to adenine at position 12 (i.e. G12A) of SEQ ID NO. 2. Nucleotide sequences comprising said polymorphism, that are suitable to identify said polymorphism in SEQ NO. 2, are presented as SEQ ID NO. 5 or SEQ ID NO. 6, or parts thereof. Optionally, the sequence to be used as a marker can be longer on either side of the modification, to ensure the sequence is unique in the genome and locates to the MMD-1 like gene.

Use of this marker for the identification and/or selection of a genic male sterile watermelon plant is also part of this invention.

The invention further relates to primers for detecting the modification in a MMD1-like gene as described herein. The primers are suitably PCR primers and complementary to the beginning and end of a DNA sequence comprising the polymorphism leading to the modification of the invention. In one embodiment, the primers are complementary to the two ends of SEQ ID NO. 5 or SEQ ID NO. 6.

The invention also relates to a probe for detecting the modification in a MMD1-like gene as described herein. The probe is an oligonucleotide that is capable of hybridizing to the DNA sequence comprising the polymorphism leading to the modification of the invention. The sequence of the probe is thus complementary to part of the MMD1-like gene comprising the polymorphism. In one embodiment, the probe is complementary to SEQ ID NO. 5 or SEQ ID NO. 6.

The invention further relates to a method for identifying and/or selecting a genic male sterile watermelon plant, comprising assaying the nucleic acids of a plant for the presence of one or more modifications in the MMD1-like gene, identifying and/or selecting a plant that homozygously comprises said modification as a genic male sterile plant, and optionally, verifying that the plant is genic male sterile. The identification of the presence of a modification in the MMD1-like gene may be performed by using the markers as defined above.

The invention further relates to a method for producing a genic male sterile watermelon plant, comprising modifying the wild type of the MMD1-like gene of this invention, wherein the modification results in an absence of functional MMD1-like protein, and said absence of functional MMD-1 protein leads to genic male sterile plants. The wild type of the MMD1-like gene of this invention is a gene that has, in order of increased preference, at least 90%, 91%, 92%, 93%, 94%, 95%, 96%, 97%, 98%, 99% or 100% sequence identity to SEQ ID NO: 2.

The present invention relates to a method for the production of a watermelon plant that is genic male sterile, said method comprising:
a) crossing a plant comprising the modified MMD1-like gene of the invention with a plant that does not comprise said modified MMD1-like gene;
b) optionally performing one or more rounds of selfing and/or crossing a plant resulting from step a) to obtain a further generation population;
c) selecting from the population a plant that homozygously comprises the modified MMD1-like gene that produces genic male sterile plants.

The present invention relates to a method for the production of a hybrid watermelon seed, said method comprising:
a) crossing a plant comprising the modified MMD1-like gene of the invention with a plant that does not comprise or is heterozygous for said modified MMD1-like gene; b) harvesting said resultant hybrid seed.

The presence of a modified MMD1-like gene leading to GMS may be detected using routine methods known to the skilled person such as RT-PCR, PCR, antibody-based assays, sequencing and genotyping assays, or combinations thereof. Such methods may be used to determine for example, a reduction of the expression of the wild type MMD1-like gene, a reduction of the expression of wild type MMD1-like protein, the presence of a modified mRNA, cDNA or genomic DNA encoding a modified MMD1-like protein, or the presence of a modified MMD1-like protein, in plant material or plant parts, or DNA or RNA or protein derived therefrom.

An example of a routinely used genotyping assay is KASP (Kompetitive Allele Specific PCR), which is based on allele-specific oligo extension. A person skilled in the art is familiar with designing and performing a KASP assay for the detection of genetic variation in plants. In the present invention, KASP markers were designed based on SEQ ID NO. 5 and SEQ ID NO. 6, in order to detect the G to A SNP mutation. An ‘A’ haplotype means that the plant comprises the wild type MMD1-like gene homozygously, a ‘B’ haplotype means that the plant comprises the mutant MMD1-like gene homozygously, and an ‘H’ haplotype means that the plant comprises the mutant MMD1-like gene heterozygously. Other genotyping techniques can also be used for the detection of the SNP that is linked to the trait of the invention.

Modifications or mutations of the wild type MMD1-like gene can be introduced randomly by means of one or more chemical compounds, such as ethyl methane sulphonate (EMS), nitrosomethylurea, hydroxylamine, proflavine, N-methly-N-nitrosoguanidine, N-ethyl-N-nitrosourea, N-methyl-N-nitro-nitrosoguanidine, diethyl sulphate, ethylene imine, sodium azide, formaline, urethane, phenol and ethylene oxide, and/or by physical means, such as UV-irradiation, fast neutron exposure, X-rays, gamma irradiation, and/or by insertion of genetic elements, such as transposons, T-DNA, retroviral elements.

Mutagenesis also comprises the more specific, targeted introduction of at least one modification by means of homologous recombination, oligonucleotide-based mutation introduction, zinc-finger nucleases (ZFN), transcription activator-like effector nucleases (TALENs) or Clustered Regularly Interspaced Short Palindromic Repeat (CRISPR) systems

Modifying the wild type MMD1-like gene could also comprise the step of targeted genome editing, wherein the sequence of the wild type MMD1-like gene is modified, or wherein the wild type MMD1-like gene is replaced by another MMD1-like gene that is modified. This can be achieved by means of any method known in the art for modifying DNA in the genome of a plant, or by means of methods for gene replacement. Such methods include genome editing techniques and homologous recombination.

Homologous recombination allows the targeted insertion of a nucleic acid construct into a genome, and the targeting is based on the presence of unique sequences that flank the targeted integration site. For example, the wild type locus of a MMD1-like gene could be replaced by a nucleic acid construct comprising a modified MMD1-like gene.

Modifying the wild type MMD1-like gene can involve inducing double strand breaks in DNA using zinc-finger nucleases (ZFN), TAL (transcription activator-like) effector nucleases (TALEN), Clustered Regularly Interspaced Short Palindromic Repeats/CRISPR-associated nuclease (CRISPR/Cas nuclease), or homing endonucleases that have been engineered to make double-strand breaks at specific recognition sequences in the genome of a plant, another organism, or a host cell.

TAL effector nucleases (TALENs) can be used to make double-strand breaks at specific recognition sequences in the genome of a plant for gene modification or gene replacement through homologous recombination. TAL effector nucleases are a class of sequence-specific nucleases that can be used to make double-strand breaks at specific target sequences in the genome of a plant or other organism. TAL effector nucleases are created by fusing a native or engineered transcription activator-like (TAL) effector, or functional part thereof, to the catalytic domain of an endonuclease, such as, for example, Fok I. The unique, modular TAL effector DNA binding domain allows for the design of proteins with potentially any given DNA recognition specificity. Thus, the DNA binding domains of the TAL effector nucleases can be engineered to recognise specific DNA target sites and thus, used to make double-strand breaks at desired target sequences.

ZFNs can be used to make double-strand breaks at specific recognition sequences in the genome of a plant for gene modification or gene replacement through homologous recombination. The Zinc Finger Nuclease (ZFN) is a fusion protein comprising the part of the Fok I restriction endonuclease protein responsible for DNA cleavage and a zinc finger protein which recognizes specific, designed genomic sequences and cleaves the double-stranded DNA at those sequences, thereby producing free DNA ends (Urnov et al, 2010, Nat. Rev. Genet. 11:636-46; Carroll, 2011, Genetics 188:773-82).

The CRISPR/Cas nuclease system can also be used to make double-strand breaks at specific recognition sequences in the genome of a plant for gene modification or gene replacement through homologous recombination. The CRISPR/Cas nuclease system is an RNA-guided DNA endonuclease system performing sequence-specific double-stranded breaks in a DNA segment homologous to the designed RNA. It is possible to design the specificity of the sequence (Jinek et al, 2012, Science 337: 816-821; Cho et al, 2013, Nat. Biotechnol. 31:230-232; Cong et al, 2013, Science 339:819-823; Mali et al., 2013, Science 339:823-826; Feng et al, 2013, Cell Res. 23:1229-1232). Cas9 is an RNA-guided endonuclease that has the capacity to create double-stranded breaks in DNA in vitro and in vivo, also in eukaryotic cells. It is part of an RNA-mediated adaptive defence system known as Clustered Regularly Interspaced Short Palindromic Repeats (CRISPR) in bacteria and archaea. Cas9 gets sequence-specificity when it associates with a guide RNA molecule, which can target sequences present in an organism's DNA based on their sequence. Cas9 requires the presence of a Protospacer Adjacent Motif (PAM) immediately following the DNA sequence that is targeted by the guide RNA. The Cas9 enzyme has been first isolated from Streptococcus pyogenes (SpCas9), but functional homologues from many other bacterial species have been reported, such as Neisseria meningitides, Treponema denticola, Streptococcus thermophilus, Francisella novicida, Staphylococcus aureus, etcetera. For SpCas9, the PAM sequence is 5′-NGG-3′, whereas various Cas9 proteins from other bacteria have been shown to recognise different PAM sequences. In nature, the guide RNA is a duplex between crRNA and tracrRNA, but a single guide RNA (sgRNA) molecule comprising both crRNA and tracrRNA has been shown to work equally well (Jinek et al, 2012, Science 337: 816-821). The advantage of using an sgRNA is that it reduces the complexity of the CRISPR-Cas9 system down to two components, instead of three. For use in an experimental setup (in vitro or in vivo) this is an important simplification.

An alternative for Cas9 is, for example, Cpf1, which does not need a tracrRNA to function, which recognises a different PAM sequence, and which creates sticky end cuts in the DNA, whereas Cas9 creates blunt ends. On the one hand, genetic modification techniques can be applied to express a site-specific nuclease, such as an RNA-guided endonuclease and/or guide RNAs, in eukaryotic cells. One or more DNA constructs encoding an RNA-guided endonuclease and at least one guide RNA can be introduced into a cell or organism by means of stable transformation (wherein the DNA construct is integrated into the genome) or by means of transient expression (wherein the DNA construct is not integrated into the genome, but it expresses an RNA-guided endonuclease and at least one guide RNA in a transient manner). This approach requires the use of a transformation vector and a suitable promoter for expression in said cell or organism. Organisms into which foreign DNA has been introduced are considered to be Genetically Modified Organisms (GMOs), and the same applies to cells derived therefrom and to offspring of these organisms. In important parts of the worldwide food market, transgenic food is not allowed for human consumption, and not appreciated by the public. There is however also an alternative, “DNA-free” delivery method of CRISPR-Cas components into intact plants that does not involve the introduction of DNA constructs into the cell or organism.

For example, introducing the mRNA encoding Cas9 into a cell or organism has been described, after in vitro transcription of said mRNA from a DNA construct encoding an RNA-guided endonuclease, together with at least one guide RNA. This approach does not require the use of a transformation vector and a suitable promoter for expression in said cell or organism.

Another known approach is the in vitro assembly of ribonucleoprotein (RNP) complexes, comprising an RNA-guided endonuclease protein (for example Cas9) and at least one guide RNA, and subsequently introducing the RNP complex into a cell or organism. In plants, the use of RNPs has been demonstrated in protoplasts, for example with polyethylene glycol (PEG) transfection (Woo et al, 2015, Nat. Biotech. 33: 1162-1164). After said modification of a genomic sequence has taken place, the protoplasts or cells can be used to produce plants that harbour said modification in their genome, using any plant regeneration method known in the art (such as in vitro tissue culture).

Breaking DNA using site specific nucleases, such as, for example, those described herein above, can increase the rate of homologous recombination in the region of the breakage. Thus, coupling of such effectors as described above with nucleases enables the generation of targeted changes in genomes which include additions, deletions and other modifications.

More recently developed genome editing techniques include base editing and prime editing, which are genome editing techniques that are capable of creating highly precise and targeted changes to the genome. In short, base editing is a genome editing technique that generates precise point mutations in genomic DNA or in cellular RNA without generating double stranded breaks, thus avoiding random insertion and deletions that are associated with DNA breaks. Prime editing is another genome editing technique that can make targeted small insertions, deletions and substitutions in a precise targeted manner and also does not require double stranded breaks to function.

Throughout this application, the words “modification” and “mutation” are used interchangeably. It is to be understood that a mutation can be a type of modification and that a modification can also be indicated as a mutation.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1

### Genetic Modification of Watermelon Seeds by Ethyl Methane Sulfonate (EMS)

Watermelon (Citrullus lanatus) seeds from an internal diploid watermelon line were treated by a mutagenic EMS treatment. A number of approximately 10000 seeds were imbibed under agitation (60 rpm) and using 1 ml/seed (150 ml per dose) with a solution of 1% (w/v) EMS during 16 hours at temperature of 28° C. After the treatment, the EMS solution was discarded and the seeds were washed/incubated in water with gently shake for 15 min. The seed wash procedure was repeated at least seven additional times.

The treated seeds were germinated in the nursery and the resulting M1 plants, approximately 5000 plants, were transplanted, grown and self-pollinated in a greenhouse to produce seeds. The resulting seeds were used as starting material M2 to identify individual plants that show mutations.

### Example 2

### Identification of Genic Male Sterile Watermelon Plants

A mutant watermelon plant, having a mutated version of the MMD1-like gene was identified in a TILLING screen of M2 plants that were created in Example 1. DNA sequencing revealed the replacement of a guanine (G) to adenine (A) at position 12 (i.e. G12A) of the nucleotide sequence in one M2 plant (SEQ ID NO. 4). This replacement or mutation translates at the protein level as a substitution of a Tryptophan amino acid (SEQ ID NO. 1) into a premature stop codon, at position 4 of the wildtype protein sequence, resulting in a truncated version of the MMD1-like protein (SEQ ID NO. 3).

Watermelon plants from the M3 generation, were assessed for the GMS phenotype and genotyped for the presence of the mutated MMD1-like gene by means of a KASP genotyping assay. KASP (Kompetitive Allele Specific PCR), is based on allele-specific oligo extension. A single-nucleotide polymorphism (SNP) occurs when a single nucleotide in a DNA sequence differs between samples. A person skilled in the art is familiar with designing and performing a KASP assay for the detection of genetic variation in plants. For the detection of the G to A SNP mutation, KASP markers were designed based on SEQ ID NO. 5 and SEQ ID NO. 6. An ‘A’ haplotype means that the plant comprises the wild type MMD1-like gene homozygously, a ‘B’ haplotype means that the plant comprises the mutant MMD1-like gene homozygously, and an ‘H’ haplotype means that the plant comprises the mutant MMD1-like gene heterozygously. Other genotyping techniques can also be used for the detection of the SNP.

Several plants comprising the mutant MMD1-like gene homozygously and scoring ‘B’ were identified. The male flowers of these mutant watermelon plants appeared to be normal in morphology and development (see FIG. 2A) but did not produce pollen at all (See FIG. 2B, 2C). The homozygous mutant plants did not set seed when self-pollinated.

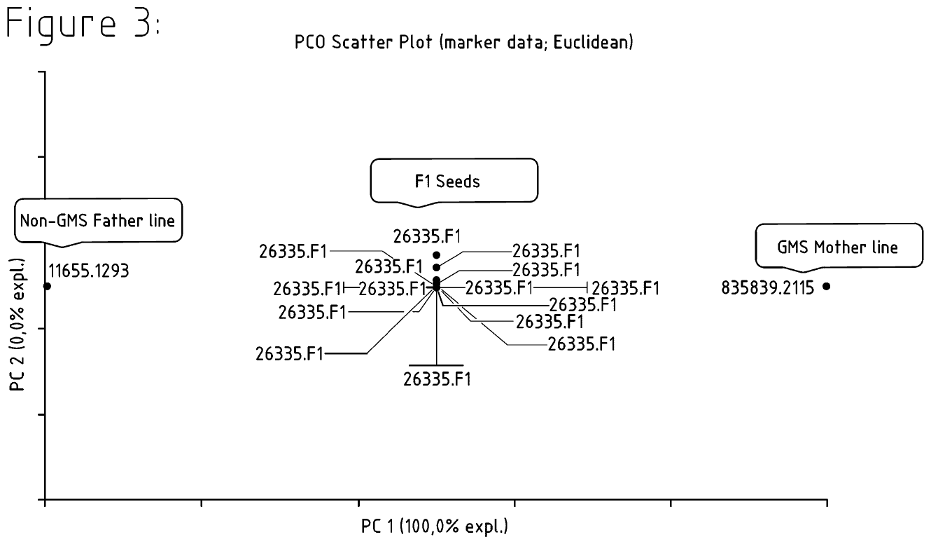

Seven of these mutant plants were transplanted into a closed tunnel alongside several plants that lacked the mutant MMD1-like gene (e.g. wild type for the MMD1-like gene, scoring ‘A’). The mutant plants were used as the female parent in the cross. Male flowers from all plants were evaluated three weeks after transplanting. Mature male flowers (See FIG. 2D) comprising pollen were present only in the wild type male parent lines (i.e. male fertile plants which lack the mutant MMD1-like gene) (See FIG. 2E, 2F). Pollination was allowed to take place using bees. Seventy-five days following transplantation, mature fruits were collected from the female parent lines. Hybrid seeds were removed from the fruit, cleaned, and sown. A panel of 83 markers that spanned across the watermelon genome were used to verify the genetic profile of the hybrid seeds. The genetic relationship (see FIG. 3) based on this panel verified that no inbred seeds were obtained from the fruits grown on the female parent line, providing further evidence that the female parent line is genic male sterile and the female fertility of the plants comprising the mutant MMD1-like gene homozygously was not impaired.

Additional trials in the years 2019-2021 were conducted with watermelon plants from the M3 generation. In addition to the self-pollination experiments of the mutant plants comprising the MMD1-like gene homozygously, the results of which are shown in Table 1, additional observations were made as follows: T19R.155-4 plants comprising the mutant MMD1-like gene heterozygously were self-pollinated, yielded fruits with seeds, and the mutant plants comprising the MMD1-like gene homozygously which were grown from these seeds were male sterile; T20R.401—male flowers of the mutant plants comprising the MMD1-like gene homozygously were used to pollinate wild type female flowers and this yielded no fruits indicating that the male flowers were genic male sterile; the female flowers of the mutant plants comprising the MMD1-like gene homozygously plants were pollinated with wild type male flowers and this yielded fruits with seeds indicating that the female flowers were fertile; T20R.417 and T21R.406: Pollen viability of 4 plants comprising the MMD1-like gene homozygously and 5 plants comprising the MMD1-like gene heterozygously was assessed on culture medium that allowed observation of pollen tubules which is an indication of pollen viability; the male flowers of the plants comprising the MMD1-like gene homozygously had normal flower development as compared to wild type male flowers but did not comprise pollen; the male flowers of the plants comprising the MMD1-like gene heterozygously had normal flower development as compared to wild type male flowers, with viable pollen observed.

TABLE 1

| | Number of Mutant | Pollen on | |
|---|---|---|---|
| | Plants (‘B’ | male | Self |
| Trial | haplotype) | flowers? | fruits? |
| T19R.115 | 3 | NO | NO |
| T19R.155 | 2 | NO | NO |
| T20R.401 | 2 | NO | NO |
| T20R.417 | 2 | NO | NO |
| T21R.406 | 2 | NO | NO |
| T21R.409 | 15 | NO | NO |
| T21R.911 | 5 | NO | NO |

### Example 3

### Triploid Watermelon Hybrid Production Using the GMS Mutant

For triploid watermelon hybrid production using GMS mutant plants, a tetraploid mutant MMD1-like gene female parent line is generated. A tetraploid mutant MMD1-like gene female parent line is generated by first selecting a diploid watermelon plant that is heterozygous for the MMD1-like gene, as determined by the KASP marker assay as described in Example 2. This plant is then treated with colchicine to allow for chromosome doubling. Following colchicine treatment, the plant is selfed for several generations, checked that the ploidy level has been maintained, and the resultant seeds are grown. Only plants comprising the MMD1-like gene homozygously, as determined by the KASP marker assay as described in Example 2, are selected and will be used as the female parent line. The obtained GMS tetraploid line is then crossed with an inbred diploid non-GMS male parent line (i.e. comprising the wild type MMD1-like gene), giving rise to triploid hybrid plants that produce triploid seedless watermelon fruits. In this way, the GMS mutant of the invention readily eliminates laborious controlled pollination practices during triploid watermelon hybrid production.

The invention is further described by the following numbered paragraphs:

1. A modified Male Meiocyte Death 1-like (MMD1-like) gene, the wild type of which comprises the nucleotide sequence of SEQ ID NO. 2, encoding a MMD1-like protein comprising one or more modifications in the wild type amino acid sequence of SEQ ID NO. 1, or in an amino acid sequence having at least 90% sequence identity to SEQ ID NO. 1.

2. The modified MMD1-like gene of paragraph 1, wherein the amino acid sequence encoded by the modified MMD1-like gene has at least 91%, 92%, 93%, 94%, 95%, 96%, 97%, 98%, or 99% sequence identity to SEQ ID NO. 1.

3. The modified MMD1-like gene of paragraph 1 or 2, wherein the modification is a premature stop codon which results in a truncated protein.

4. The modified MMD1-like gene of any one of the paragraphs 1 to 3, wherein the premature stop codon is the result of a nucleotide replacement from guanine to adenine at position 12 of SEQ ID NO. 2.

5. The modified MMD1-like gene of any one of the paragraphs 1 to 4, wherein the MMD1-like protein encoded by the modified MMD1-like gene is non-functional.

6. The modified MMD1-like gene of any one of paragraphs 1 to 5, wherein the MMD1-like protein encoded by the modified MMD1-like gene comprises SEQ ID NO. 3.

7. The modified MMD1-like gene of any one of paragraphs 1 to 6, wherein the modified protein as a result of the one or more modifications, confers genic male sterility in a plant when homozygously present.

8. A watermelon plant (Citrullus lanatus var. lanatus) comprising the modified MMD1-like gene of any one of paragraphs 1 to 7.

9. The watermelon plant of paragraph 8, wherein the modified MMD1-like gene encoding the modified MMD-1 protein when homozygously present in the genome of the plant, confers genic male sterility.

10. A watermelon seed comprising a modified MMD1-like gene of any one of paragraphs 1 to 7, wherein the plant grown from the seed is a plant of paragraph 8 or 9.

11. A progeny plant of the watermelon plant of paragraph 8 or 9, or of the plant grown from the watermelon seed of paragraph 10, wherein the progeny plant comprises the modified MMD1-like gene of any one of paragraphs 1 to 7.

12. A fruit harvested from the watermelon plant of paragraph 8 or 9, or from a watermelon plant grown from the watermelon seed of paragraph 10, wherein the fruit comprises the modified MMD1-like gene of any one of paragraphs 1 to 7.

13. Propagation material capable of developing into or being derived from the plant of paragraph 8 or 9, wherein the propagation material comprises the modified MMD1-like gene of any one of paragraphs 1 to 7, and wherein the propagation material is selected from the group consisting of a microspore, a pollen, an ovary, an ovule, an embryo, an embryo sac, an egg cell, a cutting, a root, a root tip, a hypocotyl, a cotyledon, a stem, a leave, a flower, an anther, a seed, a meristematic cell, a protoplast and a cell, or a tissue culture thereof.

14. Use of the modified MMD1-like gene of any one of paragraphs 1 to 7 for producing a genic male sterile plant.

15. Use of paragraph 14, wherein the genic male sterile plant is produced by introducing the modified MMD1-like gene into its genome, in particular by means of mutagenesis, introgression, cis-genesis, transgenesis, or combinations thereof.

16. A marker for the detection of a modified MMD1-like gene, wherein the marker comprises an oligonucleotide that detects a nucleotide replacement from guanine to adenine at position 12 of SEQ ID NO. 2.

17. The marker according to paragraph 16, comprising the sequence of SEQ ID NO. 5 or SEQ ID NO. 6, or parts thereof.

18. Use of the marker of paragraph 16 or 17, for identifying and/or selecting a watermelon plant comprising a modified MMD1-like gene.

19. A method for imparting genic male sterility in a watermelon plant, comprising introducing a modification in a MMD1-1 like gene, wherein the modification leads to a AMD1-like gene as defined in any one of paragraphs 1 to 7.

20. A method for producing a watermelon plant that is genic male sterile, comprising introducing a modification in a MMD1-like gene comprising SEQ ID NO. 1.

21. The method of paragraph 21 wherein the modification leads to a MMD1-like gene as defined in any one of paragraphs 1 to 7.

22. A method for the production of a watermelon plant which is genic male sterile, comprising:
(a) crossing the plant of paragraph 8 or 9, with another plant;
(b) optionally performing one or more rounds of selfing and/or crossing of the plant
   resulting from the cross of step a) to obtain a further generation population;
(c) selecting from the population resulting from the cross of step a), or from the further generation population of step b), a plant that comprises the modified MMD1-like gene homozygously, as defined in any one of paragraphs 1 to 7.

23. A method for the production of a hybrid seed, comprising crossing a first parent plant with a second parent plant and harvesting the resultant hybrid seed, wherein the first parent plant is the plant of paragraph 8 or 9 and the second parent plant is a plant that does not comprise or is heterozygous for the modified MMD1-like gene.

24. A hybrid seed produced by the method of paragraph 24.

25. A method for identifying and/or selecting a watermelon plant that comprises a modified MMD1-like gene of any one of paragraphs 1 to 7, comprising:
(a) assaying nucleic acids of a plant for the presence of one or modifications in the MMD1-like gene;
(b) identifying and/or selecting a plant as a plant that is genic male sterile, if one or
   more modifications in the MID-like gene are present; and
(c) optionally verifying if the plant is genic male sterile.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A watermelon (Citrullus lanatus) plant comprising a modified Male Meiocyte Death 1-like (MMD1-like) gene;
wherein SEQ ID NO: 2 is the nucleotide sequence of the wild type MMD1-like gene;
wherein the modified MMD1-like gene encodes a MMD1-like protein comprising a modification in the wild type amino acid sequence of SEQ ID NO. 1, or in an amino acid sequence having at least 95% sequence identity to SEQ ID NO. 1;
wherein:
(a) the modified MMD1-like gene comprises a premature stop codon encoding and resulting in expression of the modified MMD1-like protein lacking the PHD_MMD1_like domain;
wherein the PHD_MMD1_like domain is amino acids at position 551 to 595 of SEQ ID NO: 1;
(b) the modified MMD1-like gene comprises a substitution of a guanine with an adenine at position 12 of SEQ ID NO: 2;
(c) the modified MMD1-like gene has the sequence of SEQ ID NO: 4; or
(d) the modified MMD1-like protein has the amino acid sequence of SEQ ID NO: 3; and
the modified MMD1-like gene, when homozygously present, confers genic male sterility to the plant.

2. (canceled)

3. The plant of claim 1, the modified MMD1-like gene comprises the premature stop codon encoding and resulting in expression of the modified MMD1-like protein lacking the PHD_MMD1_like domain.

4. The plant of claim 3, wherein the premature stop codon is the result of a nucleotide substitution from guanine to adenine at position 12 of SEQ ID NO. 2.

5. The plant of claim 1, wherein the modified MMD1-like gene has the sequence of SEQ ID NO: 4.

6. The plant of claim 1, wherein the MMD1-like protein encoded by the modified MMD1-like gene has the amino acid sequence of SEQ ID NO. 3.

7. The plant of claim 1, wherein the modified MMD1-like gene is homozygously present and confers genic male sterility to the plant.

8. The plant of claim 1, wherein the modified MMD1-like gene is heterozygously present.

9. (canceled)

10. A watermelon (Citrullus lanatus) seed from, or that produces, the watermelon plant of claim 1 comprising the modified MMD1-like gene; and/or
a progeny plant of the watermelon (Citrullus lanatus) plant of claim 1, wherein the progeny plant comprises the modified MMD1-like gene; and/or
a fruit harvested from the watermelon (Citrullus lanatus) plant of claim 1, wherein the fruit comprises the modified MMD1-like gene.

11. (canceled)

12. (canceled)

13. A propagation material capable of developing into, and/or having been derived from, the plant of claim 1, wherein the propagation material comprises the modified MMD1-like gene, and wherein the propagation material comprises a microspore, a pollen, an ovary, an ovule, an embryo, an embryo sac, an egg cell, a cutting, a root, a root tip, a hypocotyl, a cotyledon, a stem, a leave, a flower, an anther, a seed, a meristematic cell, a protoplast, or a cell or a tissue culture thereof.

14. A method for producing a watermelon (Citrullus lanatus) plant that exhibits genic male sterility comprising introducing into the genome of a watermelon plant a modified Male Meiocyte Death 1-like (MMD1-like) gene;
wherein SEQ ID NO: 2 is the nucleotide sequence of the wild type ADI-like gene;
wherein the modified MMD1-like gene encodes a MMD1-like protein comprising a modification in the wild type amino acid sequence of SEQ ID NO: 1, or in an amino acid sequence having at least 95% sequence identity to SEQ ID NO: 1;
wherein:
(a) the modified MMD1-like gene comprises a premature stop codon encoding and resulting in expression of the modified MMD1-like protein lacking the PHD_MMD1_like domain;
wherein the PHD_MMD1_like domain is amino acids at position 551 to 595 of SEQ ID NO: 1;
(b) the modified MMD1-like gene comprises a substitution of a guanine with an adenine at position 12 of SEQ ID NO: 2;
(c) the modified MMD1-like gene has the sequence of SEQ ID NO: 4; or
(d) the modified MMD1-like protein has the amino acid sequence of SEQ ID NO: 3; and
the modified NADI-like gene, when homozygously present, confers genic male sterility to the plant;
wherein the modified MMD1-like gene is introduced into the genome of the watermelon (Citrullus lanatus) plant by means of mutagenesis, introgression, cis-genesis, transgenesis, or combinations thereof.

15. (canceled)

16. A marker for the detection of the modified Male Meiocyte Death 1-like (MMD1-like) gene of the watermelon plant of claim 1;
wherein the modified MMD1-like gene comprises the substitution of the guanine with the adenine at position 12 of SEQ ID NO: 2; and
the marker comprises an oligonucleotide that detects the nucleotide substitution of the guanine with the adenine at position 12 of SEQ ID NO. 2.

17. The marker according to claim 16, comprising the sequence of SEQ ID NO. 5 or SEQ ID NO. 6, or parts thereof.

18. A method of selecting the watermelon plant of claim 1 that exhibits genic male sterility comprising identifying the presence of the modified MMD1-like gene of the watermelon plant of claim 1; and selecting a plant that homozygously comprises said modified MMD1-like gene as a plant that exhibits genic male sterility.

19. The method of claim 18, wherein the identifying is performed with a marker that detects the modified MMD1-like gene having the substitution of the guanine with the adenine at position 12 of SEQ ID NO: 2.

20. A method of selecting the watermelon plant of claim 1 that exhibits genic male sterility, comprising:
a) identifying by genetic analysis the presence of the modified MMD1-like gene of the plant of claim 1 in the genome of a watermelon (Citrullus lanatus) plant; and
b) selecting a plant that comprises the modified MMD1-like gene.

21. The method of claim 20 further comprising the steps of:
c) further testing the selected plant for genic male sterility as compared to an isogenic watermelon (Citrullus lanatus) plant lacking the modified MMD1-like gene, and
d) selecting the further tested plant exhibiting genic male sterility as compared to the isogenic watermelon (Citrullus lanatus) plant lacking the modified MMD1-like gene.

22. A method for producing the watermelon plant of claim 1, comprising the steps of:
(a) crossing a first watermelon (Citrullus lanatus) parent plant whose genome comprises the MMD1-like gene of the plant of claim 1 with a second watermelon (Citrullus lanatus) parent plant to obtain an F1 population;
(b) optionally performing one or more rounds of selfing and/or crossing with a F1 watermelon (Citrullus lanatus) plant to obtain a further generation population;
(c) selecting from the population resulting from the cross of step a) or from the further generation population of step b), a watermelon (Citrullus lanatus) plant that comprises the modified MMD1-like gene.

23. A method for the production of hybrid watermelon (Citrullus lanatus) seed of the watermelon plant of claim 1, comprising:
crossing a first watermelon (Citrullus lanatus) parent plant with a second watermelon (Citrullus lanatus) parent plant and harvesting the resultant hybrid seed;
wherein the first watermelon (Citrullus lanatus) parent plant or the second watermelon (Citrullus lanatus) parent plant comprises the MMD1-like gene of the watermelon plant of claim 1.

24. A hybrid watermelon (Citrullus lanatus) seed produced by the method of claim 23.

25. The method of claim 21, wherein the second watermelon (Citrullus lanatus) parent plant comprises the modified MMD1-like gene.

26. An isolated non-naturally occurring nucleic acid molecule encoding a MMD1-like protein comprising a modification in the wild type amino acid sequence of SEQ ID NO: 1, or in an amino acid sequence having at least 95% sequence identity to SEQ ID NO: 1;
wherein SEQ ID NO: 2 is the nucleotide sequence of the wild type nucleic acid molecule;
wherein:
(a) the isolated non-naturally occurring nucleic acid molecule comprises a premature stop codon encoding and resulting in expression of the modified MMD1-like protein lacking the PHD_MMD1 like domain;
wherein the PHD_MMD1_like domain is amino acids at position 551 to 595 of SEQ ID NO: 1;
(b) the isolated non-naturally occurring nucleic acid molecule comprises a substitution of a guanine with an adenine at position 12 of SEQ ID NO: 2;
(c) the isolated non-naturally occurring nucleic acid molecule has the sequence of SEQ ID NO: 4; or
(d) the modified MMD1-like protein encoded by the isolated non-naturally occurring nucleic acid molecule has the amino acid sequence of SEQ ID NO: 3.

27. The nucleic acid molecule of claim 26, comprising the premature stop codon encoding and resulting in expression of the modified MMD1-like protein lacking the PHD_MMD1_like domain.

28. The nucleic acid molecule of claim 26, comprising the substitution of the guanine with the adenine at position 12 of SEQ ID NO: 2.

29. The nucleic acid molecule of claim 26, comprising the sequence of SEQ ID NO: 4.

30. The nucleic acid molecule of claim 26, wherein the modified MMD1-like protein encoded by said isolated non-naturally occurring nucleic acid molecule has the amino acid sequence of SEQ ID NO: 3.


SEQUENCE LISTING (6 sequences)

SEQ ID NO: 1 - protein, 604 aa, Citrullus lanatus
MPIWCTLLVHENRSFVVPLYTIEEDVKLSTKPYCDHCRWWSNHFVSKRKYHIIIPLDDRWNKRLDDGGFDLDDQTHLLHGLIHCNGFGHLLCINGIEGGSKFLCGREVMDLWDRICTNLRTRKITVEDLSKKRSMDLRLLHGVAYGHPWFGRWGYRFCRGSFGVKEHHYSRALEILSSLELDKIMHDVDETDRGREMKQIIRHYRNLSETQLITLKDLLKFMLTVKYVTTIQKKTIQPIAKSPPPSRQSLQRNKQQSLVKEKQIRYRKFANAIANMDSRWPARRLEYAAEVIVKALEEKKSDKFSHGGNGMTRQDVRDAARLHIGDTGLLDYVLKSLNNVIVGNQIVRRAVNPKTRILEYTIHELRNGVQLTEEQESTQNSEPTQTPGNDIYSDVLCVYRSIFLDYPESEMVELATQGVLDSKHFAKEWPLRDEEDHLLTFIIRLMPRLTFTQSESELKSDFMPSGEVVVLPLHTTIGEVKEAAEKALRDTYYVMEQFEVMEIENLENSEDREVIFGAAESGAELFVKGMGMDLETPLKYQGGVDTWKVRCECGAGDDDGERMVACDICEIWQHTRCCGIDDADNVPPLFVCAACCDSLGPLRI

SEQ ID NO: 2 - DNA, 1815 bp, Citrullus lanatus
  source 1..1815: mol_type = genomic DNA
atgcctatctggtgtactctactcgttcatgaaaacagaagcttcgttgttccactttacaccattgaagaggacgtgaagctctccacaaagccctactgcgatcactgccgatggtggagcaatcattttgtatcgaaaagaaaatatcatatcataatacctttggatgatcggtggaacaaacgtttagatgatggcggtttcgatcttgatgatcagactcatcttctccatggcttgattcactgcaatggcttcgggcatttgctctgcatcaatggaatcgaaggaggctccaagtttctttgcggcagagaagttatggatctttgggatagaatctgcacaaatctaagaacaaggaaaattacagttgaggatttgtccaagaaaagatcaatggatcttcgtctacttcatggggtagcatatggacatccatggtttgggaggtggggatacagattctgccgaggaagctttggcgtgaaagaacaccattacagtagagctttggaaatcctcagttctctggaactagacaagataatgcacgacgtagatgaaacagatcgaggaagagagatgaagcaaatcattcgacattatagaaatctgagtgaaacacagttgatcacattgaaagatctactgaagttcatgttgactgtaaaatacgttactacgattcagaagaaaacgattcaaccaatcgccaaatctcctcctccttccagacaatctctgcagcgaaacaagcagcaatctctagtgaaggagaagcaaatacggtacagaaaattcgccaacgcaattgctaatatggacagccgatggccggcgagacggttagaatacgcggcggaggtgattgtgaaggcactggaagagaagaaatcagacaaattcagccatggcggaaatggaatgactcgacaagacgttcgagatgctgctcgccttcacattggcgacactggattgctcgattacgttctaaaatcactgaacaatgtgatcgtagggaaccagatagttcgccgcgcagtgaacccaaaaacgcggattttagaatacacgattcacgagctcagaaatggggttcaattaacagaagaacaagaatcaacacaaaattcagaaccgactcaaactcctggcaatgacatttacagcgatgtgctatgtgtatacagaagcattttcctcgactatccagaatcagaaatggtggaattagcaacacaaggagttctcgatagcaaacattttgctaaagaatggcctctccgagacgaagaagaccatctattaacgttcattatcaggttgatgccgaggctgacttttacacagtcagaatcggagttgaagagcgatttcatgccgtccggcgaggtagtggttcttccattacacacaacgatcggagaagtgaaagaagcagccgaaaaagccctaagagacacatattacgttatggaacagttcgaggttatggagatagagaatttggagaactccgaagacagagaggtgattttcggagcggcagaatcgggagcggagttgtttgtgaaaggaatggggatggatttggagacgccattgaagtaccaagggggagttgatacctggaaagtccggtgcgagtgcggcgccggcgatgatgacggcgagaggatggtggcctgtgacatctgcgagatctggcagcacactcgctgttgcggaattgacgacgccgataacgtgccgccgttgttcgtatgcgccgcctgctgcgattcgctagggccgttgagaatttga

SEQ ID NO: 3 - skipped

SEQ ID NO: 4 - DNA, 1815 bp, Citrullus lanatus
  source 1..1815: mol_type = genomic DNA
atgcctatctgatgtactctactcgttcatgaaaacagaagcttcgttgttccactttacaccattgaagaggacgtgaagctctccacaaagccctactgcgatcactgccgatggtggagcaatcattttgtatcgaaaagaaaatatcatatcataatacctttggatgatcggtggaacaaacgtttagatgatggcggtttcgatcttgatgatcagactcatcttctccatggcttgattcactgcaatggcttcgggcatttgctctgcatcaatggaatcgaaggaggctccaagtttctttgcggcagagaagttatggatctttgggatagaatctgcacaaatctaagaacaaggaaaattacagttgaggatttgtccaagaaaagatcaatggatcttcgtctacttcatggggtagcatatggacatccatggtttgggaggtggggatacagattctgccgaggaagctttggcgtgaaagaacaccattacagtagagctttggaaatcctcagttctctggaactagacaagataatgcacgacgtagatgaaacagatcgaggaagagagatgaagcaaatcattcgacattatagaaatctgagtgaaacacagttgatcacattgaaagatctactgaagttcatgttgactgtaaaatacgttactacgattcagaagaaaacgattcaaccaatcgccaaatctcctcctccttccagacaatctctgcagcgaaacaagcagcaatctctagtgaaggagaagcaaatacggtacagaaaattcgccaacgcaattgctaatatggacagccgatggccggcgagacggttagaatacgcggcggaggtgattgtgaaggcactggaagagaagaaatcagacaaattcagccatggcggaaatggaatgactcgacaagacgttcgagatgctgctcgccttcacattggcgacactggattgctcgattacgttctaaaatcactgaacaatgtgatcgtagggaaccagatagttcgccgcgcagtgaacccaaaaacgcggattttagaatacacgattcacgagctcagaaatggggttcaattaacagaagaacaagaatcaacacaaaattcagaaccgactcaaactcctggcaatgacatttacagcgatgtgctatgtgtatacagaagcattttcctcgactatccagaatcagaaatggtggaattagcaacacaaggagttctcgatagcaaacattttgctaaagaatggcctctccgagacgaagaagaccatctattaacgttcattatcaggttgatgccgaggctgacttttacacagtcagaatcggagttgaagagcgatttcatgccgtccggcgaggtagtggttcttccattacacacaacgatcggagaagtgaaagaagcagccgaaaaagccctaagagacacatattacgttatggaacagttcgaggttatggagatagagaatttggagaactccgaagacagagaggtgattttcggagcggcagaatcgggagcggagttgtttgtgaaaggaatggggatggatttggagacgccattgaagtaccaagggggagttgatacctggaaagtccggtgcgagtgcggcgccggcgatgatgacggcgagaggatggtggcctgtgacatctgcgagatctggcagcacactcgctgttgcggaattgacgacgccgataacgtgccgccgttgttcgtatgcgccgcctgctgcgattcgctagggccgttgagaatttga

SEQ ID NO: 5 - DNA, 201 bp, Citrullus lanatus
  source 1..201: mol_type = genomic DNA
cgatcagccccgccggcccatttcgtgagaatatcagaatctttcttcaacaatgtgcagagattgaagattacagaattcaagaaaaaatgcctatctggtgtactctactcgttcatgaaaacagaagcttcgttgttccactttacaccattgaagaggacgtgaagctctccacaaagccctactgcgatcactgcc

SEQ ID NO: 6 - DNA, 201 bp, Citrullus lanatus
  source 1..201: mol_type = genomic DNA
cgatcagccccgccggcccatttcgtgagaatatcagaatctttcttcaacaatgtgcagagattgaagattacagaattcaagaaaaaatgcctatctgatgtactctactcgttcatgaaaacagaagcttcgttgttccactttacaccattgaagaggacgtgaagctctccacaaagccctactgcgatcactgcc